(19) United States
(12) Patent Application Publication
(10) Pub. No.: US 2020/0000979 A1
(43) Pub. Date: Jan. 2, 2020

(54) FLUID SUCTION DEVICE WITH ENHANCED EMPTYING PORT

(71) Applicant: Angelica Myers, Irvine, CA (US)

(72) Inventor: Angelica Myers, Irvine, CA (US)

(21) Appl. No.: 16/453,814

(22) Filed: Jun. 26, 2019

Related U.S. Application Data
(60) Provisional application No. 62/763,673, filed on Jun. 28, 2018.

(51) Int. Cl. A61M 1/00; A61M 39/20; A61M 39/26

(52) U.S. Cl. CPC A61M 1/0007; A61M 1/0003; A61M 2039/1038; A61M 39/20; A61M 39/26; A61M 1/008

(57) ABSTRACT

A medical device can include a reservoir for creating a vacuum and storing fluid, an input port for receiving the fluid from a patient, an input valve that prevents the fluid from flowing from the reservoir through the input port, and a connector. The connector can have a locking interface that mates with a needleless syringe, and a valve that opens to permit flow of the fluid through the connector when connected to the needleless syringe but closes to prevent the flow when not connected. Other aspects are described and claimed.

### CROSS REFERENCE TO RELATED APPLICATIONS

None.

### TECHNICAL FIELD

An aspect of the disclosure relates to a fluid suction device capable of use in medical applications.

### BACKGROUND

A Jackson-Pratt Drain is commonly used as a post-operative drain. The Jackson Pratt Drain has a closed low negative-pressure system with a one-way valve, collecting exudate such as pus, blood, bile, or serious drainage extracted through the surgically placed flexible tubing that deposits into a grenade-shaped reservoir bulb.

### SUMMARY

The existing Jackson Pratt Drain has a stopper that generates an unintentional broken seal or dislodgement from the perimeter of the emptying port. The broken seal releases air and exudate and is the responsible component for the unintentional collapse of the reservoir bulb which triggers a failure of the low negative-pressure system. This collapse of the reservoir bulb thus terminates the performance of the existing Jackson-Pratt Drain. These malfunctions of the existing Jackson-Pratt drain are time consuming and counterproductive for the entire health care team, and ultimately a successful patient outcome.

An improved suction device 10, as shown for example in FIG. 1, is an innovation that can include replacing the existing Jackson Pratt stopper 18 with an adaptive luer lock device 16 that connects to the existing Jackson Pratt emptying port 14 of the reservoir bulb 22.

This improvement produces a tight seal to the perimeter of the emptying port which eliminates the leakage of air and exudate, or subsequent collapse of the reservoir bulb and drainage system. By maintaining a reliable closed low negative-pressure system and one-way valve 20, the Myers Device optimizes the performance of the surgically placed flexible tubing 12 allowing an accurate extraction of exudate into the collective reservoir bulb. In addition, the reliable closed system decreases the introduction of infection to the patient's surgical site.

The Myers Device also provides a dual technique of sampling and emptying of exudate. This technique can be performed under either a sterile or non-sterile field attaching any sized intended needleless luer lock tip syringe to the adaptive luer lock device. This technique of withdrawing exudate from a reliable closed system also reduces the risk of human blood exposure such as Methyl Resistant Staphylococcus aureus (MRSA), Vancomycin Resistant Enterococci (VRE), Hepatitis, and HIV to the health care worker.

Overall, the Myers Device is an innovative idea that maintains a reliable closed low negative-pressure system. This innovative idea which improves the performance of the existing Jackson-Pratt drain offers an accurate drainage system, maintains a proper seal decreasing the introduction of infection to the patient, and provides an operative dual technique of sampling and emptying of exudate from the collecting reservoir bulb. The reliable closed system also reduces the high risk of human blood exposure to the healthcare worker enriching the performance of the Jackson-Pratt Drain.

In one aspect, a medical device includes a reservoir for creating a vacuum and storing fluid; an input port for receiving the fluid from a patient into the reservoir; an input valve that prevents any fluid (e.g., exudate, blood, air, etc.) from flowing out of the reservoir through the input port; and a connector. The connector can have a locking interface that mates with a needleless syringe, and a valve, internal to the connector, that opens to permit flow of the fluid through the connector (and out of the reservoir) when connected to the needleless syringe but closes to prevent the flow when not connected.

In another aspect, a process is described, that includes providing a medical device (e.g., a Jackson Pratt Drain) and a connector (e.g., a luer lock). The connector can have a) a locking interface that mates with a needleless syringe, and b) a valve that opens to permit flow of fluid through the connector when connected to the needleless syringe but closes to prevent the flow when not connected. The medical device can have A) a reservoir for housing fluid and creating a vacuum, B) an input port for receiving the fluid from a patient, C) an input valve that prevents the fluid from flowing from the reservoir through the input port, and D) an emptying or exit port to remove the fluid from the reservoir, and to create the vacuum. The process further includes attaching the connector to the exit port of the medical device. The process can further include attaching a needleless syringe to the connector; and extracting the fluid from the reservoir with the needleless syringe.

### BRIEF DESCRIPTION OF THE DRAWINGS

Several embodiments of the disclosure here are illustrated by way of example and not by way of limitation in the figures of the accompanying drawings in which like references indicate similar elements. It should be noted that references to “an” or “one” embodiment in this disclosure are not necessarily to the same embodiment, and they mean at least one. Also, in the interest of conciseness and reducing the total number of figures, a given figure may be used to illustrate the features of more than one embodiment of the disclosure, and not all elements in the figure may be required for a given embodiment.

FIG. 1 shows a fluid suction device, according to an embodiment.

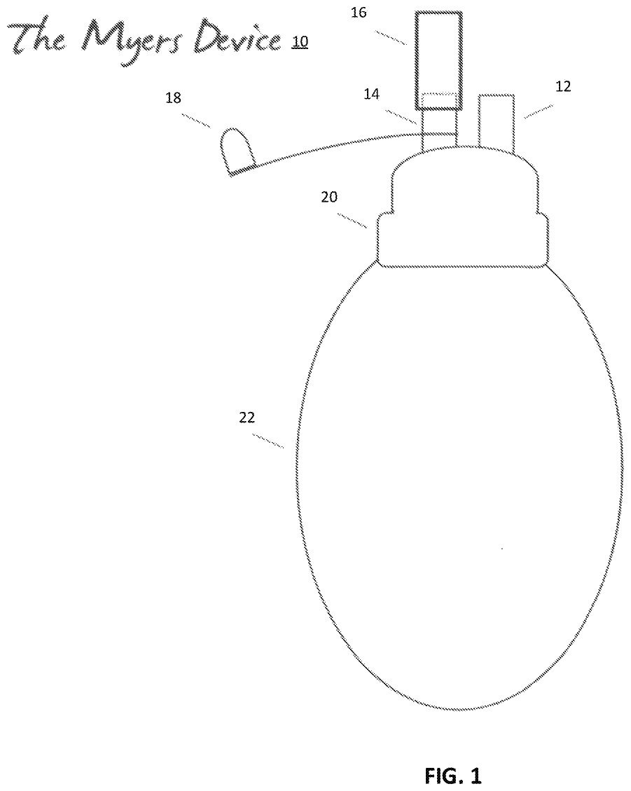

FIG. 2 shows a typical Jackson Pratt drain.

FIG. 3 shows a fluid suction device, according to an embodiment.

FIG. 4 shows a portion of a fluid suction device and a mating device, according to an embodiment.

FIG. 5 shows a process, according to one embodiment.

### DETAILED DESCRIPTION

Several embodiments of the disclosure with reference to the appended drawings are now explained. Whenever the shapes, relative positions and other embodiments of the parts described are not explicitly defined, the scope of the invention is not limited only to the parts shown, which are meant merely for the purpose of illustration. Also, while numerous details are set forth, it is understood that some embodiments of the disclosure may be practiced without these details. In other instances, well-known structures, and techniques have not been shown in detail so as not to obscure the understanding of this description.

### Typical Jackson Pratt Drain

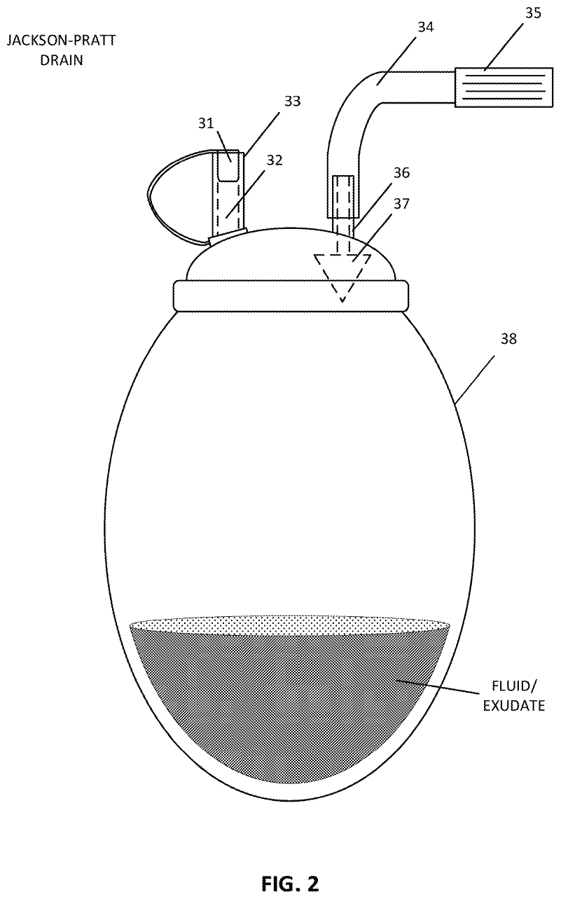

To further illustrate embodiments of the present disclosure, a typical Jackson-Pratt Drain is shown in FIG. 2. A flexible tubing 34 is attached on one side to an input port 36 of the Jackson Pratt Drain. A drain 35 can be placed in an opening of a patient (e.g., a human or animal) to extract fluid. The drain 35 can have one or more channels or perforations that help bring fluid into the flexible tubing.

A user, (e.g., a trained health care worker) can squeeze the bulb 38 to create a negative pressure (e.g., a vacuum) inside of the bulb. This vacuum will draw fluid/exudate from the patient into the bulb. A valve 37 can allow fluid to enter the bulb and prevent fluid from flowing out of the input port, to prevent potentially harmful backflow of fluid into the patient.

Typically, after the user squeezes the bulb, the stopper 31 is inserted into a channel 32 of the exit port, while the bulb is compressed. The stopper prevents fluid from flowing out of the bulb and maintains the vacuum in the reservoir caused by the compression of the bulb. As mentioned, however, the stopper can inadvertently pop loose or displace itself from the exit port because it is not securely connected to the exit port. This can result in bodily fluids spilling onto the patient and/or contaminating the site.

In addition, repetitive use of the stopper (e.g., inserting and removing the stopper repeatedly over days and weeks) can reduce the holding ability of the stopper to stay in place (e.g., due to wear and warping of the stopper or the exit port). Thus, the more the stopper is used, the less reliable it can become.

### Myers Device

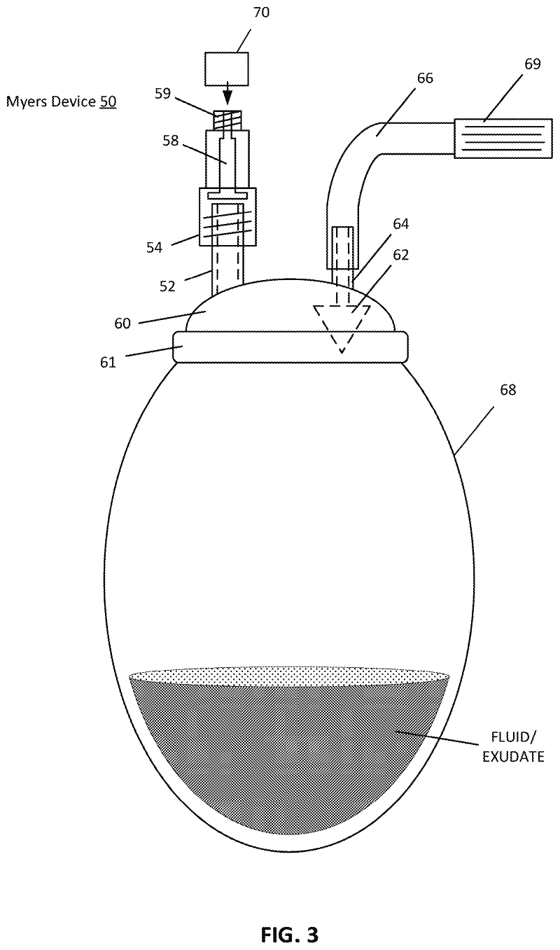

Referring now to FIG. 3, an example Myers Device 50 is shown that has a reservoir 68 for creating a vacuum and storing fluid. An input port 64 can provide an opening to the reservoir for receiving the fluid from a patient. An input valve 62 (e.g., a one-way valve) can be in line with the input port and prevent the fluid from flowing from the reservoir through the input port, but allow fluid to flow through the input port into the reservoir.

In addition to the features mentioned above, which are shared with the typical Jackson Pratt device, the Myers device has a connector 54 (e.g., a luer lock) having a locking interface 59, and a valve 58. The locking interface 59 (e.g., threads) mates with a needleless syringe and/or other compatible mating interfaces. The valve 58 opens to permit flow of the fluid through the connector when connected to for example the needleless syringe, but closes to prevent the flow when not connected. A user, (e.g., a trained health care worker) can squeeze the bulb to create a negative pressure (e.g., a vacuum) inside of the bulb. The user can then attach the connector 54 (e.g., by rotating threads of the connector onto the emptying port), which maintains the vacuum in the reservoir. Additionally or alternatively, the user can attach the needleless syringe and extract fluid from the reservoir to create or maintain the vacuum. The Myers device bulb can be emptied while simultaneously maintaining the vacuum with a needleless syringe in both a sterile field (an area kept sterile to protect patients during a medical procedure such as surgery), and a non-sterile field (for example, post-operatively such as in a recovery room or in a patient's home upon discharge). The syringe emptying process can be performed continuously, post operation, and can be performed easily due to the locking of the connector and the automatic closing of the connector through the connector valve, when the syringe is disconnected,

A lock is achieved between the needleless syringe (or other compatible mating interface) and the connector, by mating the threads of the connector onto threads of the needleless syringe. By mating with a syringe that does not have a needle, this can avoid puncture of the device (e.g., the reservoir) caused by inadvertent poking with the needle, and can make the process of extraction of fluid faster (needleless syringes have larger openings) and safer by avoiding needle stick injuries to the user.

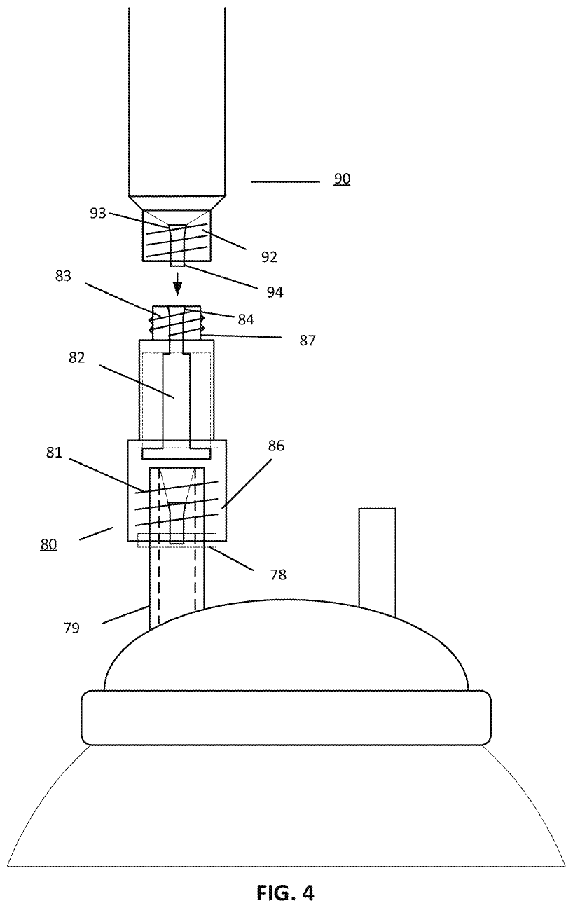

In one embodiment, the locking interface of the connector 54 is a female luer lock. A needleless syringe (e.g., having a male luer lock tip) mates with the female luer lock of the Myers Device. Luer lock fittings are securely joined by means of a tabbed hub or threads on a female luer lock which screws into threads in a sleeve on the male luer lock (see, e.g., FIG. 4). Luer lock connectors can be formed from a single mold (also known as ‘one piece luer locks’), and locking is achieved by rotating the entire luer connector or device, in this case, the Myers device and/or the syringe. In ‘two piece luer lock’, a free-rotating collar with threads is assembled to the luer, and the locking is achieved by rotating the collar. Either type of luer lock can be used in connector 54. Thus, the connector 54 can have a one piece luer lock or a two piece luer lock with free rotating collar.

Referring back to FIG. 3, in one embodiment, the connector 54 is fixed to an emptying port 52 of the medical device. The emptying port can be a tube-shaped member that provides an opening to the reservoir 68 to remove fluid, sample, and/or create a vacuum in the reservoir. The emptying port can be fixed to (or formed integrally with) a collar or cap 60 that is arranged over an opening of the reservoir. The collar and reservoir can be joined at the opening to create a hermetic seal by known methods (e.g., by an adhesive, a sealing band 61, other fastening means, or combinations thereof). The connector 54, the exit port 52, the input valve 62, and input port 64 can all be located at, fixed to, and/or integrally formed with the collar 60.

It also should be understood the connector 54 can be fixed to the emptying port 52 or it can be formed integral with (e.g., ‘built into’) the collar 60 in a monolithic manner with known manufacturing methods. Such details are not germane to the present disclosure.

The reservoir can have a variety of shapes, including ovoid (as shown in FIG. 3), spherical, cuboid, cylindrical, and more. Similarly, the reservoir can come in different sizes. For example, the reservoir can have a volumetric capacity of 100 cubic centimeters, 400 cubic centimeters, less than 800 cubic centimeters, or other capacity, determined based on application.

A flexible tube 66 can be attached to the input port 64 at a first end of the flexible tube, and to a perforated or channeled drain 69 at a second end of the flexible tube. The drain 69 can help extract fluid when inserted in a patient (e.g., through capillary action).

The Myers Device, in one embodiment, does not have a stopper that plugs into an emptying port (e.g., the manually removable stopper 31 and the tube-shaped emptying port 33 shown in FIG. 2). Instead, as shown for example in FIGS. 3 and 4, there is a connector 54 and 80 in which a valve 58 and 82 closes to prevent flow out of the emptying port 52 and 79 when the connector is not connected. The valve 82 can be spring-loaded for example formed from a springy material. When the connector is in an unconnected and ‘closed’ state, the valve can obstruct an opening 84 of the connector. The valve can be activated by compression caused by the connecting device, for example, a needleless syringe 90, or other mating connector. A member of the connecting device (e.g., stem 94) can compress and displace the valve, when connected, so that the valve no longer obstructs the opening of the connector, allowing fluid to flow back and forth into and out of the reservoir, through the connector. This can prevent spill accidents caused by inadvertent displacement of the stopper, as already described.

In one embodiment, the connector 54 has a clear housing. In this manner, the connector can be examined for residual fluid (e.g., blood, debris, etc.) and the connection between the connector 54 and a mating part (e.g., syringe) can be visually verified.

A cap 70 can be fixed onto the connector (e.g., at interface 59) when not in use and not connected to another mating device. The cap can screw onto threads of the connector or have a pressure fitting. The cap can be an anti-microbial cap having one or more anti-microbial substances on the cap.

Referring now to FIG. 4, a connector 80 includes a male luer lock portion 86 that mates to an emptying port 79 of the reservoir. The emptying port can have a tube shape. In one embodiment, the emptying port has threads that mate with the threads 81 of the connector. Alternatively, the emptying port can be smooth, without threads, and the threads 81 of the connector can simply grip onto the outside surface of the emptying port, securing the connection through friction and clamping force. An elastic and resilient ring 78 (e.g., formed from silicone, rubber, or other suitable material) can be located between threads 81 and the emptying port, in some cases, to further secure the connection between the connector and the emptying port.

A locking interface (e.g., a female luer lock) of the connector can connect to the needleless syringe 90. It should be understood, however, that instead of the needleless syringe there can be another device capable of mating to the connector 80, for example, a male luer lock. The male luer lock can be connected to other objects on a second end such as a flexible tube that allows remote fluidic access (e.g., sampling and fluid extraction) to the device.

A valve 82, housed in the connector, can open or become activated through compression caused by a stem portion 94 of the needleless syringe (or other male luer lock fitting) when connected. The valve 82 can be a compressible spring-like material. The threads 93 mate with threads 83 of the connector, achieving a locked connection. A tapered stem 94 of the needleless syringe can mate with a tapered or non-tapered opening 84 of the connector when the threads are mated. It should be understood that the connector 80 and mechanical features thereof (such as the tapered opening, the valve, the threadings, etc.) are shown for illustration purposes rather than to show an exact reproduction or geometry. Furthermore, designs of such features are known and can vary. For example, connectors with a male luer lock portion 86 and female luer lock portion 87 can be found, for example, through manufacturers such as Baxter (Clearlink Needle-free IV Access System) and CareFusion (MaxPlus® clear needleless connector) of this disclosure.

In one embodiment, an article of manufacture includes a reservoir for creating a vacuum and housing fluid; an input port for receiving the fluid from a patient; an input valve that prevents the fluid from flowing from the reservoir through the input port; and a luer lock connector (e.g., a female luer lock connector) that permits flow of the fluid through the luer lock connector (e.g., out of the reservoir) when connected to a mating luer lock device, but prevents the flow when not connected. The luer lock connector can have a valve, as described in other sections. Luer lock connectors have a locking mechanism (e.g., a threads) to secure or ‘lock’ connections between a male and female luer lock. In contrast, a Luer slip connector, also known as slip tip fittings, simply conform to Luer taper dimensions and are pressed together and held by friction. They do not have threads and can be less secure than luer locks.

### Process

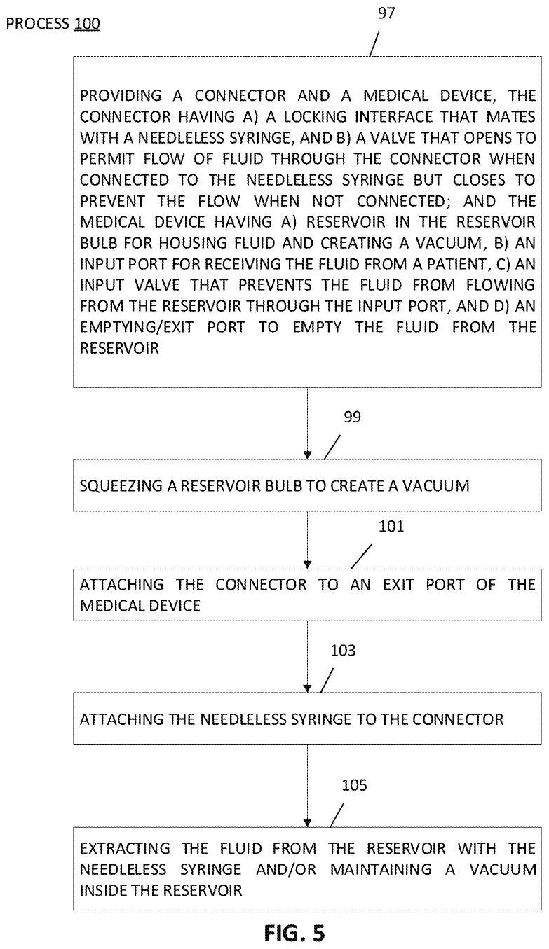

A process 100 is shown in FIG. 5 showing one aspect of the present disclosure. The process or method includes, at block 97, providing a connector (e.g., a luer lock) and a medical device (e.g., a Jackson Pratt Drain or equivalent device), where the connector has a) a locking interface that mates with a needleless syringe, and b) a valve that opens to permit flow of fluid through the connector when connected to the needleless syringe but closes to prevent the flow when not connected. The medical device has A) reservoir in the reservoir bulb for housing fluid and creating a vacuum, B) an input port for receiving the fluid from a patient, C) an input valve that prevents the fluid from flowing from the reservoir through the input port, and D) an emptying port to empty the fluid from the reservoir.

At block 99, the process can include squeezing a reservoir bulb to create a vacuum. It should be noted that this step is optional, given that the vacuum can be created in the reservoir by use of a needleless syringe, as shown in block 103.

At block 101, the process can include attaching the connector to an exit port of the medical device.

The process can further include, at block 103, attaching the needleless syringe to the connector. At block 105, the process can further include extracting the fluid from the reservoir with the needleless syringe and/or maintaining a vacuum inside the reservoir.

While certain embodiments have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. It should also be understood that while some features are shown in the figures with certain embodiments, those features can be combined with other embodiments described in this disclosure and/or shown in different figures.

What is claimed is:

1. A medical device comprising:
a reservoir for creating a vacuum and storing fluid from a patient;
an input port through which the fluid is to flow into the reservoir;
an input valve that prevents the fluid from flowing out of the reservoir through the input port; and
a connector having
a locking interface that mates with a needleless syringe, and
a valve that opens to permit flow of the fluid out of the reservoir through the connector when connected to the needleless syringe and closes to prevent the flow when not connected.

2. The medical device of claim 1, wherein the locking interface has threads and a lock is achieved between the needleless syringe and the connector by mating the threads of the connector onto threads of the needleless syringe.

3. The medical device of claim 1, wherein the locking interface is a female luer lock, and the needleless syringe has a male luer lock that mates with the female luer lock.

4. The medical device of claim 1, wherein the connector is fixed to an emptying port of the reservoir.

5. The medical device of claim 1, wherein
the connector includes a male luer lock that mates to an emptying port of the reservoir, and
the locking interface of the connector is a female luer lock that connects to the needleless syringe.

6. The medical device of claim 5, wherein an elastic ring is located between threads of the male luer lock and the emptying port.

7. The medical device of claim 1, wherein the connector, the input valve, and the input port are located on a collar that attaches to an opening of the reservoir.

8. The medical device of claim 7, wherein the connector is integral to the collar or fixed to an emptying port that is integral to the collar.

9. The medical device of claim 1, wherein the reservoir has an ovoid shape.

10. The medical device of claim 1, wherein a flexible tube is attached, at a first end, to the input port, and at a second end, to a perforated or channeled drain.

11. The medical device of claim 1, wherein a connection between the connector and the needleless syringe is leak-proof.

12. The medical device of claim 1, wherein the valve opens through compression caused by a stem portion of the needleless syringe when the needleless syringe becomes connected to the connector.

13. The medical device of claim 1, wherein the reservoir has a volumetric capacity of 100 cubic centimeters or 400 cubic centimeters or less than 800 cubic centimeters.

14. The medical device of claim 1, wherein the connector has a clear housing.

15. The medical device of claim 1, wherein the medical device does not have a removable stopper that plugs into a tube-shaped emptying port of the medical device.

16. The medical device of claim 1, wherein a tapered stem of the needleless syringe fits into a tapered opening of the connector when mated.

17. The medical device of claim 1, wherein the connector has fixed onto it, an anti-microbial cap, when not connected.

18. A system, comprising
a reservoir for creating a vacuum and housing fluid;
an input port for receiving the fluid from a patient;
an input valve that prevents the fluid from flowing from the reservoir through the input port; and
a luer lock connector that permits flow of the fluid into or out of the reservoir through the luer lock connector when connected to a mating luer lock device, and prevents the flow when not connected.

19. A method, comprising:
providing a connector and a medical device, wherein
the connector includes a) a locking interface that mates with a needleless syringe, and b) a valve that opens to permit flow of fluid through the connector when connected to the needleless syringe and closes to prevent the flow when not connected, and
the medical device includes A) a reservoir for housing fluid and creating a vacuum, B) an input port for receiving the fluid from a patient, C) an input valve that prevents the fluid from flowing from the reservoir through the input port, and D) an exit port to empty the fluid from the reservoir; and
attaching the connector to the exit port of the medical device.

20. The method of claim 19, further comprising:
attaching the needleless syringe to the connector; and
extracting the fluid from the reservoir with the needleless syringe.
